(19) United States
(12) Patent Application Publication
(10) Pub. No.: US 2013/0291234 A1
(43) Pub. Date: Oct. 31, 2013

(54) SALT TOLERANCE SYDBSP GENE DERIVED FROM SYNECHOCYSTIS, AND USES THEREOF

(71) Applicants: Jang Ryol Liu, Daejeon (KR); Suk Weon Kim, Daejeon (KR); Jong Hyun Kim, Daejeon (KR); Sung Ran Min, Daejeon (KR); Won Joong Jeong, Daejeon (KR); Myung Suk Ahn, Daejeon (KR); Young Min Park, Daejeon (KR); Myung Jin Oh, Daejeon (KR); Ji Hyun Park, Daejeon (KR)

(72) Inventors: Jang Ryol Liu, Daejeon (KR); Suk Weon Kim, Daejeon (KR); Jong Hyun Kim, Daejeon (KR); Sung Ran Min, Daejeon (KR); Won Joong Jeong, Daejeon (KR); Myung Suk Ahn, Daejeon (KR); Young Min Park, Daejeon (KR); Myung Jin Oh, Daejeon (KR); Ji Hyun Park, Daejeon (KR)

(73) Assignee: KOREA RESEARCH INSTITUTE OF BIOSCIENCE AND BIOTECHNOLOGY, Daejeon (KR)

(21) Appl. No.: 13/882,168

(22) PCT Filed: Oct. 13, 2011
(86) PCT No.: PCT/KR11/07606

§ 371 Date: Jul. 8, 2013

(30) Foreign Application Priority Data
Oct. 27, 2010 (KR) 10-2010-0105471

(51) Int. Cl. C07K 14/405; C12Q 1/68; C12N 15/82

(52) U.S. Cl. CPC C07K 14/405; C12N 15/8273; C12Q 1/6895
USPC 800/306; 530/370; 536/23.7; 435/320.1; 435/252.31; 435/252.33; 435/252.34; 435/254.21; 435/348; 435/366; 435/358; 435/352; 435/365; 435/369; 435/370; 435/357; 435/350; 435/353; 435/468; 800/298; 435/6.12; 800/317.3

(57) ABSTRACT

The present invention relates to a gene encoding Synechocystis putative DNA binding stress protein (SyDBSP protein) derived from cyanobacteria Synechocystis PCC6906; a method for enhancing the salt tolerance of a plant comprising transforming a plant cell with a recombinant vector comprising the SyDBSP gene and overexpressing the SyDBSP gene; a plant having enhanced salt tolerance produced by the aforementioned method, and seed of the plant.

### TECHNICAL FIELD

The present invention relates to a Synechocystis putative DNA binding stress protein (SyDBSP) gene derived from cyanobacteria Synechocystis; a method of enhancing the salt tolerance of a plant by overexpressing said SyDBSP gene in a plant; a plant having enhanced salt tolerance produced by said method, including seeds of the genetically modified plant.

### BACKGROUND ART

Algae, including Synechocystis, have been developed as useful sources of genes, bioenergy (e.g., Rhodophyta ethanol and seaweed biodiesel), and biomaterials (e.g., Rhodophyta pulp). Recently, seaweed biotechnology has broadened its application range to include studies of seaweed as bioreactors. These seaweed bioreactors have been used for the development and production of pharmaceutically or industrially useful proteins or substances.

In addition, several reports have shown successful applications of a gene found in Synechocystis related to agreeculture. More specifically, it has been reported that a gene derived from Synechocystis has positive effects on the enhancement of stress tolerance (i.e., salt tolerance), and also has effects for improving plant plant yields, when genetically introduced to a plant. For example, it has been reported that histidine kinase and cognate response regulators of Synechocystis sp. PCC6906, regulate the expression of hyperosmotic stress- and salt-inducible genes. These results demonstrate that not only the properties of a plant can be improved, but also the possibility of having a plant with improved productivity (e.g., producing useful substances). If a useful gene, derived from Synechocystis, is introduced to a plant, the benefits of achieving commercialization would be highly desirable.

Unlike other fresh water species, Synechocystis PCC6906 is a seawater Synechocystis species. As such, it is believed to have a well-developed mechanism of sensitivity or tolerance to salt stress. These attributes are indicative of many inherent genes present within Synechocystis PCC6906 that are related to the regulation and tolerance of salt stress.

Irrigation used for crop cultivation causes increased concentrations of water-soluble salts like sodium, calcium, magnesium, potassium, sulfate, and chloride ions. When these salts reach a certain level in soil, the root-mediated ability to absorb water is impaired in crops, and furthermore causes plant cells to have challenged metabolic activities. Following decreased water absorption by plants due to salt concentration increases, the productivity of crops decrease and the crop(s) may perish.

Not surprisingly, crop production in an irrigated field is at least three fold higher than a non-irrigated field, and the frequency of irrigation in field tends to gradually increase. Hence, continuous irrigation leads to increased salt concentration in soil, which eventually adversely affects crop productivity and subsequently leads to increased application of seed and fertilizers. Although crops with high salt-tolerance can be cultivated, they are economically unfavorable due to their high purchase cost. If costs are used for purchasing expensive salt-tolerant plants, then poorly irrigated fields result, and those poorly irrigated fields have severe soil decomposition. This chain of events may cause food shortages. In all, salinification damage is one of the most difficult problems to be solved within the agriculture community, setting significant limitations on crop productivity. According to the U.S. Dept. of Agriculture, among agricultural fields all over the world, almost 10 million hectares disappear annually due to salinification caused by irrigation. In efforts to solve salinification problems within the agricultural community, many scholars have tried to develop salt-tolerant crops based on inbreeding or outcrossing mating systems, but no clear results have come to fruition.

As a result, new techniques for inducing salt-tolerance in major crops and plants is desired. Hence, many researchers are conducting studies to enhance salt-tolerance by transforming plants and crops with foreign genes.

### DISCLOSURE OF THE INVENTION

### Problem to be Solved by the Invention

The present invention is devised under the detrimental circumstances described above, and the inventors of the present invention have isolated a SyDBSP gene, derived from Synechocystis, and have found that salt tolerance in plants can be enhanced by overexpression of the SyDBSP gene in plants.

### Means for Solving Problem

The present invention provides a Synechocystis putative DNA binding stress protein (SyDBSP protein) derived from Synechocystis PCC6906. The present invention further provides a SyDBSP gene that encodes the SyDBSP protein. The present invention further provides a recombinant vector comprising the SyDBSP gene. The present invention further provides a host cell transformed with the recombinant vector. The present invention further provides a method for enhancing the salt tolerance of a plant comprising transforming a plant cell with the recombinant vector and overexpressing the SyDBSP gene. The present invention further provides a transformed plant having enhanced salt tolerance, which is produced by the method of the invention. The present invention further provides seed of the transformed plants. The present invention further provides a composition for enhancing the salt tolerance in plants comprising a SyDBSP gene. The present invention further provides a primer set for amplifying the SyDBSP gene.

### Advantageous Effect of the Invention

According to the present invention, salt tolerance in plants can be enhanced by overexpression of a SyDBSP gene in plants. A transformed plant having enhanced salt tolerance is expected to be particularly useful in Korea, where many claimed lands and mountain slopes are present.

### BRIEF DESCRIPTION OF DRAWINGS

FIG. 1 illustrates the nucleotide sequence of the SyDBSP gene.

FIG. 2 illustrates the amino acid sequence derived from the SyDBSP gene.

FIG. 3 illustrates (A) the genetic relationships and (B) the homology between amino acid sequences derived from the SyDBSP genes of various Synechocystis.

FIG. 4 illustrates the transformation vector used in the present invention.

FIG. 5 illustrates (A) PCR results for selecting SyDBSP-transformed tobacco; (B) Southern analysis; (C) Real-time PCR results showing gene expression levels.

FIG. 6 illustrates (A) Real-time PCR results showing gene expression levels of Arabidopsis thaliana transformed with the SyDBSP gene; (B) Salt tolerance tests (i.e., chlorophyll content after NaCl treatment).

FIG. 7 illustrates (A) PCR results for selecting SyDBSP-transformed duckweed (Lemnaceae); (B) Salt tolerance tests (i.e., survival and differentiation of transformed Lemnaceae in medium containing 100 mM NaCl).

FIG. 8 illustrates (A) PCR results for selecting SyDBSP-transformed poplar (Populus alba); (B) Real-time PCR results showing gene expression levels.

FIG. 9 illustrates results of salt tolerance tests of SyDBSP-transformed poplar: (A) salt tolerance of transformed poplar using the SyDBSP gene in a NaCl containing medium; (B) results of shoot generation by transformed poplar using the SyDBSP gene in a NaCl containing medium; (C-1) Differences in salt tolerance between wild-type (WT) and transformed plants; (C-2) Fv/Fm test values of transformed poplar using the SyDBSP gene (24 hours post-treatment with 450 mM NaCl solution).

FIG. 10 illustrates changes in chlorophyll content in transformed poplar using the SyDBSP gene.

### BEST MODE(S) FOR CARRYING OUT THE INVENTION

In order to achieve the object of the invention, the present invention provides a Synechocystis putative DNA binding stress protein (SyDBSP protein)_derived from Synechocystis PCC6906 cyanobacteria, which consists of the amino acid sequence of SEQ ID NO: 2.

Included within the scope of the invention, are functional equivalents of the SyDBSP protein with SEQ ID NO: 2, isolated from Synechocystis PCC6906. As used herein, the term “functional equivalent” is intended to mean the result of an addition, substitution or deletion of amino acid residues, whereby the resulting amino acid sequence has at least 70%, preferably at least 80%, more preferably at least 90%, most preferably at least 95% homology with the amino acid sequence of SEQ ID NO: 2. The resulting functionally equivalent protein has substantially the same physiological activity as the protein described by SEQ ID NO: 2.

In another embodiment, the present invention provides a gene that encodes the above-described SyDBSP protein. The gene according to the present invention includes both genomic DNA and cDNA that encode the SyDBSP protein. Preferably, the gene according to the present invention comprises a nucleotide sequence represented by SEQ ID NO: 1. In another embodiment, a variant of SEQ ID NO: 1 is also contemplated. More specifically, in another embodiment the gene variant comprises a nucleotide sequence which has preferably at least 70%, more preferably at least 80%, still more preferably at least 90%, most preferably at least 95% sequence homology with the nucleotide sequence of SEQ ID NO: 1. Said “% sequence homology” for a certain polynucleotide is identified by comparing two optimally aligned regions. In this regard, part of the polynucleotide within the aligned or comparative regions may comprise an addition or a deletion of a nucleotide (i.e., a gap) compared to the reference sequence (i.e., without any addition or deletion).

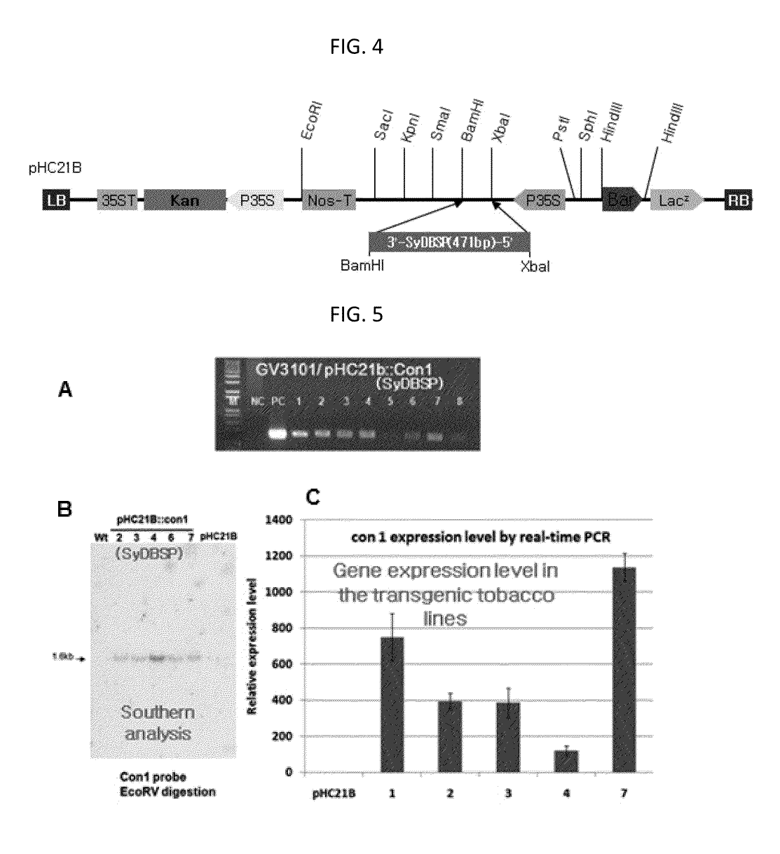

In another embodiment, the present invention provides a recombinant vector comprising a SyDBSP gene. Said recombinant vector is preferably a recombinant plant expression vector. More preferably, said recombinant vector is a transformation vector having a cleavage map as shown in FIG. 4.

As used herein, the term “recombinant” is intended to mean a cell with an ability to replicate heterogeneous nucleotides or expresses a nucleotide, peptide, heterogeneous peptide, or protein encoded by a heterogeneous nucleotide. Recombinant cells can express a gene or a gene fragment, that is not found naturally within cells, in forms such as sense or antisense strands. In addition, a recombinant cell can express a gene that is found naturally, provided that said gene is modified and re-introduced into the cell by artificial means.

The term “vector” is used herein to refer to DNA fragments and nucleotides that are delivered to a cell. A vector can be used for the replication of DNA and be independently reproduced in a host cell. The terms “delivery system” and “vector” are often interchangeably used. The term “expression vector” means a recombinant DNA molecule comprising a desired coding sequence and other appropriate nucleotide sequences that are essential for the expression of the operably-linked coding sequence in a specific host organism.

In one embodiment, the vector of the present invention can be constructed to be a vector for cloning or expression, in general. In another embodiment, the vector of the present invention can be constructed to be a vector that employs a prokaryotic cell or a eukaryotic cell as a host. For example, when the vector of the present invention is an expression vector and employs a prokaryotic cell as a host, the vector generally comprises a strong promoter which effectively promotes transcription, including, but not limited to, pLλ promoter, trp promoter, lac promoter, T7 promoter, tac promoter, and the like, a ribosome binding site for initiation of translation, and termination sequences for transcription and translation. When E. coli is employed as a host cell, the promoter and operator regions involved in E. coli tryptophan biosynthesis and the pLλ promoter can be used as regulatory sites.

In one embodiment, the vector of the present invention can be constructed by using a plasmid. Representative plasmids can include, but are not limited to, pSC101, ColE1, pBR322, pUC8/9, pHC79, pGEX series, pET series, pUC19, and the like. In another embodiment, the vector of the present invention can be constructed by using a phage. Representative phage can include, but are not limited to, λgt4·λB, λ-Charon, λΔz1, M13, and the like. In another embodiment, the vector of the present invention can be constructed by using a virus. Representative viruses can include, but are not limited to, SV40, and the like.

In another embodiment, the vector of the present invention is an expression vector and employs a eukaryotic cell as a host, a promoter originating from a mammalian genome, or a promoter originating from a mammalian virus. Representative examples of promoters from a mammalian genome include, but are not limited to, metallothionein promoter, and the like. Representative examples of a promoter originating from a mammalian virus include, but are not limited to, adenovirus late promoter, vaccinia virus 7.5K promoter, SV40 promoter, cytomegalovirus promoter, tk promoter of HSV, and the like. As a transcription termination sequence, a polyadenylated sequence is generally employed.

In one embodiment, the plant expression vector of the present invention, can employ a promoter including, but not limited to, CaMV 35S, actin, ubiquitin, pEMU, MAS, histone promoter, and the like. The term “promoter” means a DNA molecule to which RNA polymerase binds in order to initiate its transcription, and it corresponds to a DNA region upstream of a structural gene. The term “plant promoter” indicates a promoter which can initiate transcription in a plant cell. The term “constitutive promoter” indicates a promoter which is active in most of environmental conditions and development states or cell differentiation states.

With regard to transcription terminators, any conventional terminator can be used. Example of transcription terminators include, but are not limited to, nopaline synthase (NOS), rice α-amylase RAmyl A terminator, phaseoline terminator, terminator for the octopine gene of Agrobacterium tumefaciens, rrnB1/B2 terminator of E. coli, and the like.

In one embodiment, the vector of the present invention comprises an antibiotic-tolerant gene as a selectable marker. Examples of antibiotic-tolerant genes as selectable markers include, but are not limited to, genes tolerant to ampicillin, gentamycin, carbenicillin, chloramphenicol, streptomycin, kanamycin, geneticin, neomycin, tetracyclin, claforan, and the like.

In another embodiment, the present invention provides a host cell that is transformed with a recombinant vector of the present invention. Host cells, as known in the art, with the ability for stable and continuous cloning and expression of the vector of the present invention can be used. Representative examples of host cells include, but are not limited to, Bacillus sp. strains including, but not limited to, E. coli JM109, E. coli BL21, E. coli RR1, E. coli LE392, E. coli B, E. coli X 1776, E. coli W3110, and the like, Bacillus subtillus, Bacillus thuringiensis, and intestinal bacteria including, but not limited to, Salmonella typhimurium, Serratia marcescens, various Pseudomonas sp., and the like.

In another embodiment, when a eukaryotic cell is transformed with the vector of the present invention, representative examples of host cells that can be used include, but are not limited to, Saccharomyce cerevisiae cells (insect cells), human cells, CHO (Chinese hamster ovary) cells, W138 cells, BHK cells, COS-7 cells, HEK 293 cells, HepG2 cells, 3T3 cells, RIN cells, MDCK cells, and the like.

In another embodiment, when the host cell is a prokaryotic cell, delivery of the vector of the present invention into the host cell can be carried out by the CaCl₂ method as described in Cohen, S. N. et al. (1973) Proc. Natl. Acad. Sci., USA, 9:2110-2114, which is hereby incorporated by reference in its entirety, Hanahan's method as described in Hanahan, D. (1983) J. Mol. Biol., 166:557-580, which is hereby incorporated by reference in its entirety, or by an electroporation method as described in Dower, W. J. et al. (1988) Nucleic. Acids Res., 16:6127-6145, which is hereby incorporated by reference in its entirety, and the like. In another embodiment, when a host cell is a eukaryotic cell, the vector can be introduced to the host cell by a microinjection method as described in Capecchi, M. R. (1980) Cell, 22:479, which is hereby incorporated by reference in its entirety, a calcium phosphate precipitation method as described in Graham, F. L. et al. (1973) Virology, 52:456, which is hereby incorporated by reference in its entirety, an electroporation method as described by Neumann, E. et al. (1982) EMBO J., 1:841, which is hereby incorporated by reference in its entirety, a liposome-mediated transfection method as described in Wong, T. K. et al. (1980) Gene, 10:87, which is hereby incorporated by reference in its entirety, a DEAE-dextran treatment method as described by Gopal, T. V. (1985) Mol. Cell Biol., 5:1188-1190, which is hereby incorporated by reference in its entirety, or a gene bombardment method as described in Yang, N. S. et al. (1990) Proc. Natl. Acad. Sci., USA, 87:9568-9572, which is hereby incorporated by reference in its entirety, and the like.

In another embodiment, the present invention further provides a method for enhancing salt tolerance of a plant comprising transforming a plant cell with the recombinant vector and overexpressing the SyDBSP gene.

Plant transformation means any method by which DNA is delivered to a plant. The plant transformation method does not necessarily need a period for regeneration and/or tissue culture. Transformation of plant species is now quite general not only for dicot plants but also for monocot plants. A person having ordinary skill in the art can employ any transformation method used for introducing a hybrid DNA as in the present invention to appropriate progenitor cells. Representative methods of transformation include, but are not limited to, a calcium/polyethylene glycol method for protoplasts as described in Krens, F. A. et al. (1982) Nature, 296:72-74 and Negrutiu I. et al. (1987) Plant Mol. Biol., 8:363-373, both of which are hereby incorporated by reference in their entirety, an electroporation method for protoplasts as described in Shillito R. D. et al. (1985) Bio. Technol., 3:1099-1102, which is hereby incorporated by reference in its entirety, a microscopic injection method for plant components as described in Crossway A. et al. (1986) Mol. Gen. Genet., 202:179-185, which is hereby incorporated by reference in its entirety, a particle bombardment method for various plant components (i.e., DNA or RNA-coated) as described in Klein T. M. et al. (1987) Nature, 327:70, which is hereby incorporated by reference in its entirety, or a (non-complete) viral infection method in a Agrobacterium tumefaciens-mediated gene transfer by plant invasion or transformation of fully ripened pollen or microspore as described in EP 0 301 316, which is hereby incorporated by reference in its entirety, and the like. The preferred method for the present invention includes Agrobacterium mediated DNA transfer. More specifically, a so-called binary vector technique as disclosed in EP A 120 516 and U.S. Pat. No. 4,940,838, both of which are hereby incorporated by reference in their entirety, can be preferably adopted for the present invention.

The transformation according to the invention may be mediated by Agrobacterium tumefaciens. Further, the method of the present invention comprises regenerating a transformed plant from the transformed plant cells, as described above. As for the method of regenerating a transformed plant from transformed plant cells, any method well known in the pertinent art can be used.

In order to achieve yet another purpose of the present invention, the present invention provides a transformed plant with enhanced salt tolerance that is produced by the method of the present invention.

More specifically, salt tolerant plants according to the present invention can be obtained by transforming a plant with the recombinant vector containing the SyDBSP gene. The progeny of the transformed plants (i.e., including shoots and roots) are also provided for in the present invention. In one embodiment, a fragment of a plant transformed with the recombinant SyDBSP vector is applied on a suitable medium and the plant is cultivated under suitable conditions to induce shoot formation. Once shoots are formed, they are subsequently cultivated in a hormone-free medium. After two weeks, shoots are then transferred to a medium for inducing root formation. Following root induction, the plants are then planted in soil for acclimation, yielding the progeny of a salt tolerant plant.

The present invention further provides seed of the plants with enhanced salt tolerance.

The present invention further provides a transformed plant with enhanced salt tolerance according to transformation with the vector of the present invention.

In one embodiment of the method, the plant used can be either a monocot or a dicot plant. Examples of monocot plants include, but are not limited to, rice, wheat, barley, bamboo shoot, corn, taro, asparagus, onion, garlic, scallion, leek, wild rocambole, hemp, ginger, duckweed, and the like. Examples of dicot plants include, but are not limited to, tobacco, Arabidopsis thaliana, eggplant, pepper, tomato, potato, burdock, crown daisy, lettuce, Chinese bellflower, chard, spinach, sweet potato, celery, carrot, coriander, parsley, Chinese cabbage, cabbage, leaf mustard radish, watermelon, melon, cucumber, zucchini, gourd, strawberry, soy bean, mung bean, kidney bean, sweet pea, poplar, and the like. More preferably, the dicot plants are tobacco, Arabidopsis thaliana, poplar, or duckweed.

The present invention further provides a composition for enhancing salt tolerance of a plant, in which the composition comprises the SyDBSP gene. The composition of the present invention comprises the SyDBSP gene as an effective component, whereby introducing the SyDBSP gene to a plant and allowing it to express therein, salt tolerance of the plant can be enhanced. In one embodiment, the composition of the present invention, the SyDBSP gene, may preferably consist of the nucleotide sequence of SEQ ID NO: 1. In other embodiments, the SyDBSP gene may include those in which certain base sequences are inserted, substituted, or deleted within the sequence of the SyDBSP gene.

As used herein, the term “salt tolerance” means an ability of certain kind of a plant to grow under osmotic stress or stress that is caused by the salt or ion content present in water and soil. For example, when moisture is supplied (i.e., irrigation) containing a mixture of water and ions, which is disadvantageous for the growth of similar plant-types, or when moisture is supplied as a medium containing ions for cultivation, a plant exhibiting an increased growth rate compared to a plant of a similar type and/or variant type, the plant is said to have salt tolerance.

The present invention still further provides a primer set for amplifying the SyDBSP gene. In one embodiment, the primer set consists of oligonucleotides having SEQ ID NO: 3 and SEQ ID NO: 4.

According to the present invention, the term “primer” indicates a single-stranded oligonucleotide which is complementary to the nucleotide strand to be copied and it can function as an initiation point for the synthesis of primer elongation product. The length and the sequence of the above-described primer should satisfy the condition required for the initiation of the synthesis of an elongation product. In one embodiment, an oligonucleotide used as a primer may comprise a nucleotide analogue including, but not limited to, a phosphorothioate, an alkyl phosphorothioate, a peptide nucleic acid, or an intercalating agent.

Provided herein are non-limiting examples used to illustrate how those of ordinary skill in the art may make and use the present invention. These examples are not intended to limit the scope of the invention as contemplated by the inventors. Amounts, temperatures, and times are approximate.

### EXAMPLES

Experimental Methods

1. Construction of Nuclear Transformation Vector

The SyDBSP gene of Synechocystis PCC6906 was obtained from genomic DNA of Synechocystis PCC6906 by PCR amplification using primer 5′-gctctagaATGACTTCAATTAATATCGGTATT-3′ (primer SEQ ID NO: 3, XbaI site is marked with underline) and 5′-cgggatccCTATTTGTTCAGAACCCGGAGCAT-3′ (primer SEQ ID NO: 4, BamHI site is marked with underline). The amplified gene was cloned into the TA cloning vector (Solgent, Korea) to confirm the base sequence. The SyDBSP gene with a confirmed base sequence was digested with XbaI/BamHI, subcloned into pHC21B, and named pHC21B-SyDBSP. The gene insertion direction was confirmed by fragment size obtained by restriction enzyme digestion and PCR results. Each plant was then transformed with the vector for incorporating the SyDBSP gene.

2. Plant Transformation and Culture Condition

2-1. Tobacco Nuclear Transformation and Culture Condition

Agrobacterium GV3101 was transformed with the pHC21B-SyDBSP transformation vector according to a freeze-thaw method. A single colony was inoculated into YEP medium containing 100 mg/L rifampicin and 50 mg/L kanamycin and cultured for ˜2 days (28° C., in a dark shaking incubator). A tobacco leaf cultured in an incubator was cut to give an explant with a size of about 5×5 mm² (excluding a peripheral part) and then allowed to float on 10 mL Agro solution, which had been diluted to O.D 0.4-0.6, such that the stomata face in an upward direction. It was then co-cultured under dark conditions for approximately 2 days. The explant after co-culture was washed twice with sterilized water and once with a solution containing 500 mg/L carbenicillin or claforan. After removing moisture with a sterilized paper towel, it was planted on a medium for shoot regeneration (MS+2 mg/L (or 1 mg/L) BA+0.1 mg/L NAA+500 mg/L carbenicillin or claforan+100 mg/L kanamycin) such that the stomata face in an upward direction, and then cultured for 16 hours at 25° C. under daylight conditions. Three to four weeks after culture, the shoots regenerated from the leaf explant were cut and transferred to a MS medium (MS+500 mg/L carbenicillin or claforan+100 mg/L kanamycin) to form roots. Then, rooted plants were transplanted in soil and grown under controlled greenhouse conditions for plant growth (Phytotron).

2-2. Arabidopsis thaliana Transformation and Culture Condition

Seeds of Arabidopsis thaliana were subjected to a low-temperature treatment (4° C.) for 4 days under dark conditions and then sown into soil. About four weeks later, transformation was performed according to a vacuum infiltration method using Agrobacterium tumefaciens GV3101 containing a salt-tolerant gene. Seeds of the transformed Arabidopsis thaliana were selected on MS selection medium (1/2MS+0.5 g/L MES+10 g/L sucrose+50 mg/L kanamycin, 100 mg/L cefotaxime) containing kanamycin as a selection marker, and only the homozygotes were selected and used for the experiment. Every culture was performed at 25° C. under about 80 μmol m⁻² s⁻¹ cool-white fluorescence conditions with light cycles of 16 hours.

2-3. Lemnaceae Transformation and Culture Condition

Transformation of duckweed (Lemnaceae) was performed by using thalloid leaves of Lemnaceae and Agrobacterium. Specifically, as a medium for culturing thalloid of Lemnaceae, the concentration of all inorganic salts in the MS medium was reduced by half and the thalloid was cultured on a medium (1/2MS 1BA medium) containing 1 mg/L BA, 0.4 mg/L thiamine HCl, 100 mg/L myoinositol, 30 g/L sucrose, and 4 g/L Gelrite. It was then induced to have individual growth while being cultured under light culture conditions (about 80 μmol m⁻² s⁻¹, with light/dark cycles of 16/8 hours) at 25° C.

Using a knife, a scratch was created in the cultured thalloid of Lemnaceae. Then the thalloid was immersed for 20 minutes in a bacterial solution with Agrobacterium tumefaciens GV3101, which had been transformed with a salt tolerant gene. After the infection, Agrobacterium tumefaciens was removed and the dried thalloid leaves were transferred to a solid medium for plant culture containing 100 μM Acetosyringone, and co-cultured in a dark room for 72 hours at 25° C. The co-cultured leaves were washed three to four times using a broth containing 300 mg/L carbenicillin to fully remove surface adhered Agrobacterium. After drying for 10 to 20 minutes, the leaves were transferred to a selection medium containing 250 mg/L kanamycin and 300 mg/L carbenicillin as a selection marker. The differentiated thalloid leaves, after planting onto the selection medium, were then subjected to subculture with three-week intervals.

2-4. Poplar Transformation and Culture Condition

For poplar transformation, nodal segments were isolated from a poplar (Populus alba X P. tremula var. glandulosa) and used. Specifically, nodal segments of a 4-week old poplar were infected for 20 min with Agrobacterium tumefaciens which had been cultured in LB liquid medium containing 150 μM Acetosyringone. The infected nodal segments were washed with a 0.85% NaCl solution and then added onto filter paper to remove residual Agrobacterium tumefaciens. The washing process was repeated three times and the segments were then cultured in CIM medium (MS, 1 mg/L 2,4-D, 0.1 mg/L NAA, 0.01 mg/L BA, pH 5.8) containing no antibiotics for two days in a 24° C. incubator. Thereafter, the cultured nodal segments were transferred to CIM medium (MS, 50 mg/L kanamycin, 300 mg/L cefotaxime, 1 mg/L 2,4-D, 0.1 mg/L NAA, 0.01 mg/L BA, pH 5.8) and callus formation was induced for three to four weeks. Once the callus was formed from the nodal segments, it was transplanted to SIM medium (WPM, 50 mg/L kanamycin, 300 mg/L cefotaxime, 1 mg/L zeatin, 0.1 mg/L BA, 0.01 mg/L NAA, pH 5.5) and shoot formation was induced for ˜8 weeks. The induced shoots were transplanted in RIM medium (MS, 50 mg/L kanamycin, 300 mg/L cefotaxime, 0.2 mg/L IBA) to induce root formation.

From the leaf tissues of the plant having roots that were induced in RIM medium (MS, 50 mg/L kanamycin, 300 mg/L cefotaxime, 0.2 mg/L IBA), genomic DNA was extracted and the transformed plant was selected by using PCR.

The selected poplar with induced root formation was removed from the medium. After washing the medium adhered onto the roots with distilled water, the plant was transplanted to appropriately wetted soil, which had been previously sterilized and kept in a sealed container, while being careful not to hurt the roots. The container was sealed again and the plant was allowed to grow for ˜20 days in a 24° C. incubator (cultured under light conditions for 16 hours, and cultured under dark conditions for 8 hours).

3. Southern Analysis

Total genomic DNA was isolated from the leaves of the transformed plant by using a DNeasy Plant Mini Kit (Qiagen, Hilden, Germany). About 4 μg of the genomic DNA was digested with EcoRV (for the tobacco transformant), subjected to electrophoresis with 1% agarose gel, and transferred to a Zeta-Probe GT Blotting Membrane (Bio-Rad, Hercules, Calif.). From the genome of Synechocystis PCC6906, a fragment of about 500 bp was amplified by PCR using 5′-TCGGTATTCCTGAAGCTGATCGCA-3′ primer (SEQ ID NO: 5) and 5′-ATCCGACGCTAAAGAAGTGGTGGA-3′ primer (SEQ ID NO: 6). After labeling with a radioactive element [³²P] dCTP, insertion of the SyDBSP gene was confirmed. Pre-hybridization and hybridization were performed for 16 hours at 65° C. in a 0.25 M sodium phosphate buffer (pH 7.2) containing 7% (w/v) SDS. After two washings at 65° C. for 30 minutes each with a 0.2 M sodium phosphate buffer (pH 7.2) containing 5% (w/v) SDS, a reaction on an X-ray film was allowed to occur for three hours followed by confirmation.

4. Real-Time PCR

By using Trizol Reagent (GIBCOBRL, N.Y., USA), total RNA was extracted from the leaves of tobacco and poplar. cDNA was synthesized by using 5 μg of the total RNA, oligo dT₁₅, and a M-MLV Reverse Transcriptase (Enzynomics, Daejeon, Korea)

Seeds of Arabidopsis thaliana were sterilized and allowed to form sprouts on MS medium (1/2MS, 0.5 g/L MES, 10 g/L sucrose, 100 mg/L cefotaxime). From the Arabidopsis thaliana cultured for 7 days under 40 μmol m⁻² sec⁻¹ cool-white fluorescent light conditions with light cycles of 16 hours at 25° C., total RNA was extracted by using an RNeasy Mini kit (QIAGEN, Hilden, Germany) and an RNase-Free DNase Set (QIAGEN, Hilden, Germany). cDNA was synthesized by using 6 μg of the total RNA, oligo dT₁₅, and a M-MLV Reverse Transcriptase (Enzynomics, Daejeon, Korea).

The synthesized cDNA of tobacco, Arabidopsis thaliana, and poplar was subjected to Real-time PCR by using a SolGent™ Real-time PCR kit (Solgent, Daejeon, Korea) and a DNA Engine Opticon 2 (MJ Research, Waltham, USA).

Each primer sequence used for the real time PCR is given in the Table 1 below.

TABLE 1

| | | | SEQ |
|---|---|---|---|
| | | | ID |
| No. | Name | Base sequence | NO: |
| 1 | primer-F for SyDBSP amplification | 5′- | 3 |
| | and PCR determination | gctctagaATGACTTCAATTAATATCGGTATT- | |
| | | 3′ | |
| 2 | primer-R for SyDBSP amplification | 5′- | 4 |
| | and PCR determination | cgggatccCTATTTGTTCAGAACCCGGAGCA | |
| | | T-3′ | |
| 3 | SyDBSP Southern-F | 5′-TCGGTATTCCTGAAGCTGATCGCA-3′ | 5 |
| 4 | SyDBSP Southern -R | 5′-ATCCGACGCTAAAGAAGTGGTGGA-3′ | 6 |
| 5 | SyDBSP Real-Time PCR primer-F | 5′-TCGGTATTCCTGAAGCTGATCGCA-3′ | 7 |
| 6 | SyDBSP Real-Time PCR primer-R | 5′-TGGAAGTTGTGGGTCTGGAGGTAA-3′ | 8 |
| 7 | Tobacco control Real-Time primer-F | 5′-AAGGAGTGTCCCAATGCTGAGTGT-3′ | 9 |
| 8 | Tobacco control Real-Time primer-R | 5′-TCACCACCAGCCTTCTGGTAAACA-3′ | 10 |
| 9 | Arabidopsis thaliana control Real- | 5′-TTTGACCGGAAAGACCATCACCCT-3′ | 11 |
| | Time primer-F | | |
| 10 | Arabidopsis thaliana control Real- | 5′-AAGACGCAGGACCAAGTGAAGAGT-3′ | 12 |
| | Time primer-R | | |
| 11 | Poplar control Real-Time primer-F | 5′-TGCAGGCATCCACGAAACCACATA-3′ | 13 |
| 12 | Poplar control Real-Time primer-R | 5′-GGCTAGTGCTGAGATTTCCTTGCT-3′ | 14 |

5. Measurement of Chlorophyll Content

For Arabidopsis thaliana, 500 g of 95% ethanol was added to thirty plants which had been cultured for 5 days with light cycles of 16 hours/8 hours in MS medium containing 1% sucrose. For poplar, 2 ml of 95% ethanol was added to leaf pieces (1.13 cm²), which were then cultured for 18 hours under dark conditions at 4° C., and the chlorophylls were then extracted. The extracted chlorophylls were measured for OD_{664.2} and OD_{648.6} using a spectrophotometer to determine the content of chlorophyll A and chlorophyll B. Chlorophyll content was expressed as sum of chlorophyll A and chlorophyll B.

6. Determination of Salt Tolerance

For Arabidopsis thaliana, 50 sterilized seeds were cultured to MS medium containing 1% sucrose and allowed to sprout by culturing for 5 days. After transferring them to MS medium containing NaCl at specific concentrations and 1% sucrose, the plant was then allowed to grow for 5 days with light and dark cycles of 16 hours:8 hours (light:dark). Thirty plants were then subjected to a chlorophyll measurement test for measuring the change in chlorophyll content thereby investigating the salt tolerance of the transformed Arabidopsis thaliana.

For Lemnaceae, the transformed plant was sub-cultured with three-week intervals. The resulting differentiated plant was added to MS medium containing NaCl at specific concentrations. Thereafter, survival and differentiation of the plant was compared to those of a control group to determine the salt tolerance.

For poplar, primary salt tolerance was investigated based on shoot formation by a transformed plant compared to a control group plant on a medium for shoot generation containing NaCl at specific concentrations. Three weeks after acclimating the transformed plant in soil, it was immersed in a 300 mM NaCl solution for 24 hours. While being allowed to recover in 0.1% Hyponex solution, the change in Fv/Fm values and chlorophyll content was measured using a Handy PEA (Hansa Tech, USA) apparatus to determine the salt tolerance of the transformed plant. For selecting a plant showing the best salt tolerance among the salt tolerant poplar transformants, a 24-hour treatment with a 450 mM NaCl solution was performed eight weeks after the acclimation, and then the change in Fv/Fm values and chlorophyll content was measured and compared to those of the control group.

Other experimental methods that are not described herein were performed according to commonly used methods for plant cultivation, seed selection, and other molecular biology techniques.

### EXAMPLES

### Example 1

### SyDBSP Gene Derived from Synechocystis PCC 6906

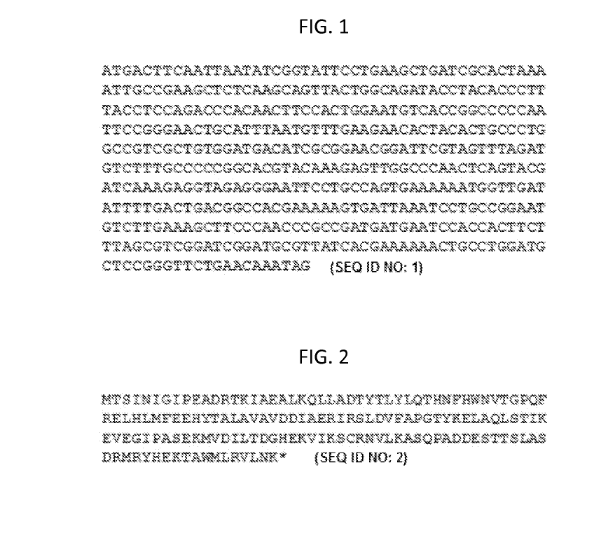

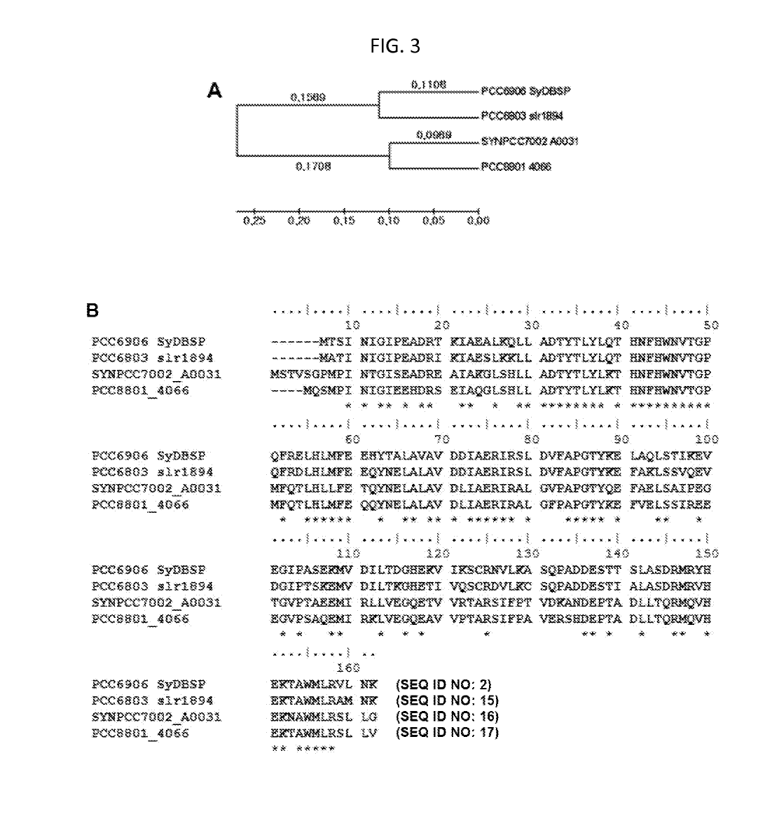

The nucleotide sequence of the SyDBSP gene derived from Synechocystis PCC6906 was determined by isolating the genome of Synechocystis PCC6906, and obtaining the entire nucleotide sequence information using GS-FLX (Roche, USA). The SyDBSP gene consists of 471 nucleotides and encodes a sequence consisting of 156 amino acids (FIG. 1 and FIG. 2). The amino acid sequence of the SyDBSP gene of Synechocystis PCC6906 exhibited the closest genomic relationship with freshwater inhabiting Synechocystis PCC6803 slr1894 (i.e., an 80% identity and 91% positive relationship). In addition, it also exhibited a close genomic relationship with the genes of Cyanothece sp. PCC8801_{—}4066 (FIG. 3).

### Example 2

### Vector for Transformation with SyDBSP Gene Derived from Synechocystis PCC6906 and Selection of Transformed Plants

For producing a transformed plant, the SyDBSP gene from the Synechocystis PCC6906 genome was amplified by using primer 5′-gctctagaATGACTTCAATTAATATCGGTATT-3′ (SEQ ID NO: 3, XbaI site is marked with underline) and primer 5′-cgggatccCTATTTGTTCAGAACCCGGAGCAT-3′ (SEQ ID NO: 4, BamHI site is marked with underline) followed by digestion with restriction enzymes. XbaI/BamHI site of pHC21B was cut using a restriction enzyme and a SyDBSP gene fragment was inserted thereto to produce a nuclear transformation vector (FIG. 4). The plant introduced with the transformation vector was selected on a medium containing kanamycin, and the selected transformed plant was subjected to PCR using the primers of the SyDBSP gene to confirm the insertion of the SyDBSP gene. Further, expression of the SyDBSP gene in each transformed plant was followed by Real-time PCR to determine the level of expression.

### Example 3

### Production of Transgenic Tobacco Plants with a SyDBSP Gene Derived from Synechocystis PCC6906

From the transformed tobacco plant introduced with the SyDBSP gene derived from Synechocystis PCC6906, seeds of a TO generation were obtained and sterilized. By selecting a plant exhibiting tolerance in MS medium containing 3% sucrose and 50 mg/L kanamycin, seeds of a T1 generation were obtained. A plant was generated from the obtained seeds, and following isolation of genomic DNA, subsequent PCR, and final Southern Analysis, the incorporation of the SyDBSP gene was confirmed. Total RNA was then isolated and expression levels of the introduced gene were determined by Real-time PCR. As shown in FIG. 5A, a total of six transformants were found to have gene insertion among eight tobacco plants transformed with the SyDBSP gene. In addition, among those transformed plants, five of them were found to have single copy insertions of the gene (FIG. 5B). Lastly, overexpression of the SyDBSP gene in the transformed tobacco plant was confirmed as a result of Real-time PCR analysis (FIG. 5C).

### Example 4

### Salt Tolerance of Transgenic Arabidopsis thaliana Plants with SyDBSP Gene Derived from Synechocystis PCC6906

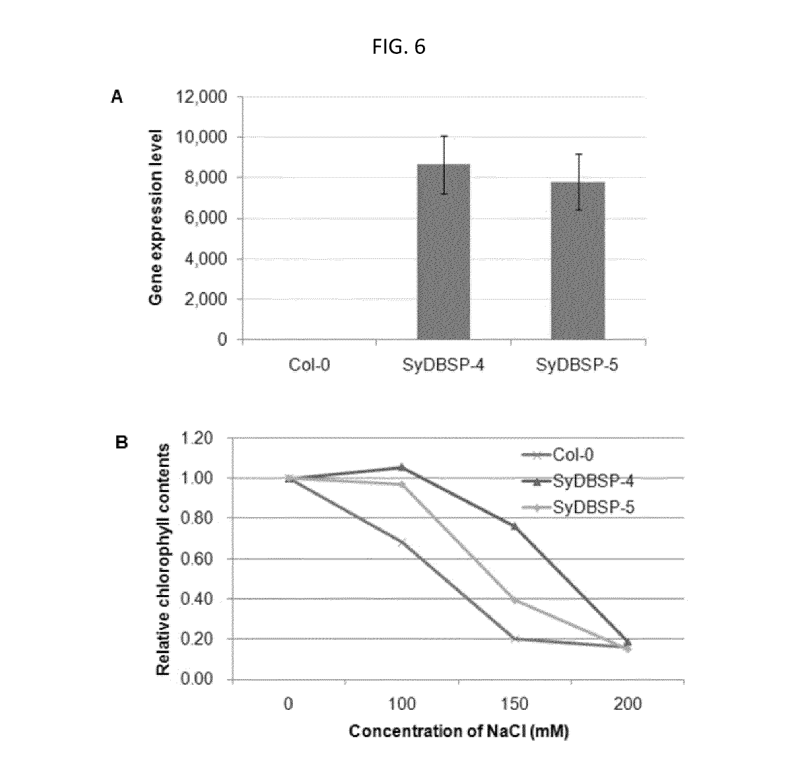

T1 seeds of Arabidopsis thaliana transformed with the SyDBSP gene were sterilized and added to a medium containing 1% sucrose. After being subjected to a low-temperature treatment (4° C.) for 4 days under dark conditions, they were cultured for 5 days at 25° C. under 80 μmol m⁻² s⁻¹ cool-white fluorescence conditions with light cycles of 16 hours. From the cultured plants, total RNA was isolated and expression levels of the SyDBSP gene were determined using Real-time PCR. In two independent lines (SyDBSP-4 and SyDBSP-5), overexpression of the SyDBSP gene was confirmed (FIG. 6A).

The SyDBSP-4 and SyDBSP-5 lines were added to a medium containing 1% sucrose followed by low-temperature treatment and culture for 5 days under the conditions described above. Both lines were then transferred to MS medium containing NaCl at concentrations of 100, 150, or 200 mM, and cultured for 7 days under the same conditions. A determination of chlorophyll content after extraction from thirty plants found relatively high chlorophyll content from the transformed Arabidopsis thaliana in a medium containing NaCl at concentrations of 100 mM or 150 mM compared to Col-0 as a control group (FIG. 6B). This result indicates that the transformed Arabidopsis thaliana overexpressing the SyDBSP gene has gained tolerance to NaCl (i.e., salt tolerance).

### Example 5

### Salt Tolerance of Transgenic Lemnaceae Plants with the SyDBSP Gene Derived from Synechocystis PCC6906

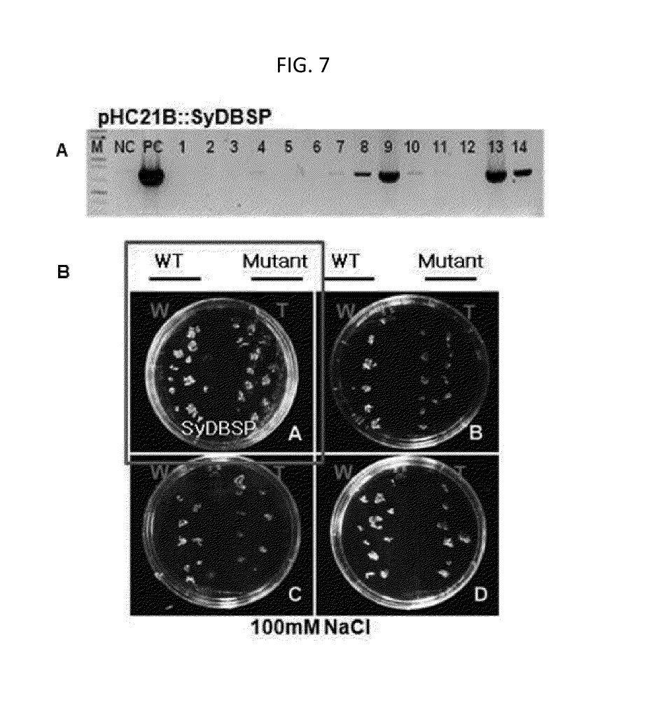

For selection of Lemnaceae transformed plants with the SyDBSP gene, in which the transformed Lemnaceae has been produced according to a tissue culture method for Lemnaceae, genomic DNA was extracted from the transformed Lemnaceae. According to PCR analysis, four transformed plants were obtained (FIG. 7A).

The transformed plants were sub-cultured with three week intervals for inducing differentiation. For salt tolerance measurements, the plants were added to MS medium containing 100 mM NaCl and cultured at 25° C. under 80 μmol m⁻² s⁻¹ cool-white fluorescence conditions with light cycles of 16 hours, and their survival and differentiation were observed and recorded. Results indicated that the control group had perished in the medium containing NaCl, but the transformed Lemnaceae with the SyDBSP gene survived, and showed ongoing differentiation (FIG. 7B, left panel: control group (WT), right panel: transformed Lemnaceae (Mutant)).

### Example 6

### Salt Tolerance of Transgenic Poplar Plants with the SvDBSP Gene Derived from Synechocystis PCC6906

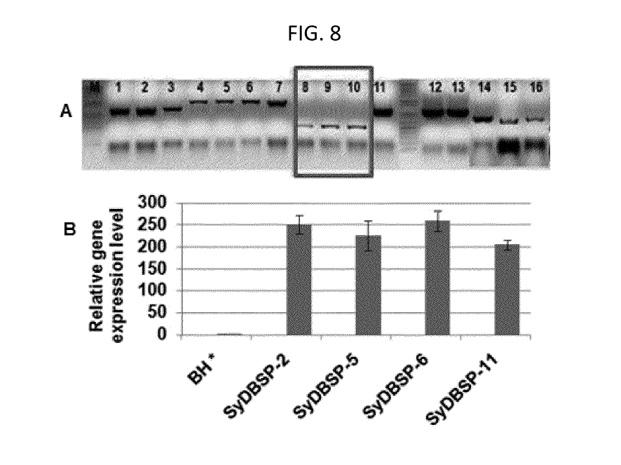

In order to select the transformed poplar introduced with the SyDBSP gene, PCR was performed using the SyDBSP primer (FIG. 8A). From the selected transformed poplar, genomic DNA was isolated and expression levels of the introduced gene were determined by Real-time PCR. Results confirmed that overexpression occurs in the transformed poplar (FIG. 8B).

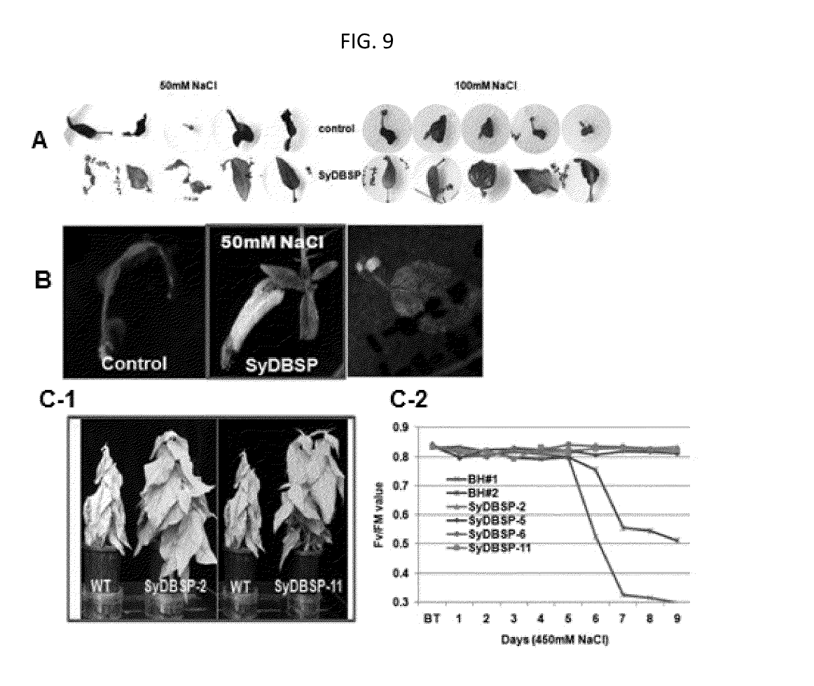

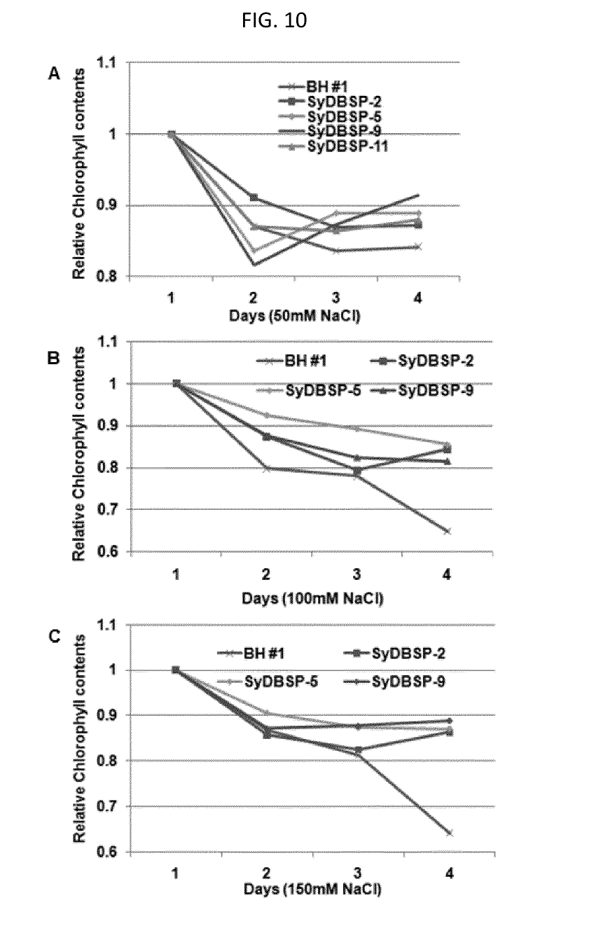

In order to confirm salt tolerance in the transformed poplar, leaves of each transformed poplar were added to SIM medium for shoot generation (WPM, 50 mg/L kanamycin, 300 mg/L cefotaxime, 1 mg/L zeatin, 0.1 mg/L BA, 0.01 mg/L NAA, pH 5.5) containing 50 mM or 100 mM NaCl. Then the leaves were observed with regards to survival and shoot generation, while being cultured for 8 weeks at 25° C. under light conditions. Results showed that the leaves of the transformed poplar survived in the medium containing 100 mM NaCl, and shoots were generated in the same medium containing 50 mM NaCl. Alternatively, the plants of the control group perished at both concentrations of NaCl (FIGS. 9A and B). Eight weeks after acclimation in soil, the transformed poplar was exposed to a 450 mM NaCl solution for 24 hours. Following irrigation with 0.1% Hyponex solution at 2-day intervals, the Fv/Fm values were measured. Results of the measurements show, with BT as a control group, rapid decrease of the Fv/Fm values five days after the NaCl solution treatment. However, the poplar transformed with the SyDBSP gene continuously maintained normal values of ˜0.8 (FIG. 9C-2). Furthermore, three weeks after the acclimation period, a leaf disk with a size of about 1.13 cm² was prepared from the leaves of the transformed poplar and immersed in a NaCl solution at concentrations of 50, 100, or 150 mM to measure changes in chlorophyll content. Results indicated at each concentration, contrary to the control group showing rapid decreases in chlorophyll content, the SyDBSP transformed poplar maintained relatively high chlorophyll content (FIG. 10). As such, it was found that the transformed poplar having the SyDBSP gene derived from Synechocystis PCC6906 exhibited excellent salt tolerance compared to the control group.

1. A Synechocystis putative DNA binding stress protein (SyDBSP) derived from Synechocystis PCC 6906 which consists of the amino acid sequence of SEQ ID NO: 2.

2. A gene that encodes the SyDBSP protein of claim 1.

3. The gene according to claim 2, wherein the gene consists of a nucleotide sequence of SEQ ID NO; 1.

4. A recombinant vector comprising the gene of claim 2.

5. A host cell transformed with the recombinant vector of claim 4.

6. A method for enhancing salt tolerance of a plant, comprising:
transforming a plant cell with the recombinant vector according to claim 4; and
overexpressing the gene.

7. A transformed plant having enhanced salt tolerance produced by the method of claim 6.

8. The transformed plant according to claim 7, wherein the plant is a dicot plant or a monocot plant.

9. The transformed plant according to claim 7, wherein the plant is tobacco, Arabidopsis thaliana, duckweed, or poplar.

10. Seed of the plant of claim 7.

11. A transformed plant having enhanced salt tolerance produced by transformation with the recombinant vector of claim 4.

12. A composition for enhancing salt tolerance of plant comprising the gene of claim 2.

13. A primer set for amplification of the gene of claim 2, the primer set consisting of oligonucleotides of SEQ ID NO: 3 and SEQ ID NO: 4.


SEQUENCE LISTING (14 sequences)

SEQ ID NO: 1 - DNA, 471 bp, Synechocystis PCC6906
atgacttcaattaatatcggtattcctgaagctgatcgcactaaaattgccgaagctctcaagcagttactggcagatacctacaccctttacctccagacccacaacttccactggaatgtcaccggcccccaattccgggaactgcatttaatgtttgaagaacactacactgccctggccgtcgctgtggatgacatcgcggaacggattcgtagtttagatgtctttgcccccggcacgtacaaagagttggcccaactcagtacgatcaaagaggtagagggaattcctgccagtgaaaaaatggttgatattttgactgacggccacgaaaaagtgattaaatcctgccggaatgtcttgaaagcttcccaacccgccgatgatgaatccaccacttctttagcgtcggatcggatgcgttatcacgaaaaaactgcctggatgctccgggttctgaacaaatag

SEQ ID NO: 2 - protein, 156 aa, Synechococcus PCC6906
MTSINIGIPEADRTKIAEALKQLLADTYTLYLQTHNFHWNVTGPQFRELHLMFEEHYTALAVAVDDIAERIRSLDVFAPGTYKELAQLSTIKEVEGIPASEKMVDILTDGHEKVIKSCRNVLKASQPADDESTTSLASDRMRYHEKTAWMLRVLNK

SEQ ID NO: 3 - DNA, 32 bp, Artificial Sequence
  SyDBSP PCR primer-F
gctctagaatgacttcaattaatatcggtatt

SEQ ID NO: 4 - DNA, 32 bp, Artificial Sequence
  SyDBSP PCR primer-R
cgggatccctatttgttcagaacccggagcat

SEQ ID NO: 5 - DNA, 24 bp, Artificial Sequence
  SyDBSP Southern-F primer
tcggtattcctgaagctgatcgca

SEQ ID NO: 6 - DNA, 24 bp, Artificial Sequence
  SyDBSP Southern-R primer
atccgacgctaaagaagtggtgga

SEQ ID NO: 7 - DNA, 24 bp, Artificial Sequence
  SyDBSP Real-Time PCR primer-F
tcggtattcctgaagctgatcgca

SEQ ID NO: 8 - DNA, 24 bp, Artificial Sequence
  SyDBSP Real-Time PCR primer-R
tggaagttgtgggtctggaggtaa

SEQ ID NO: 9 - DNA, 24 bp, Artificial Sequence
  Tobacco control Real-Time primer-F
aaggagtgtcccaatgctgagtgt

SEQ ID NO: 10 - DNA, 24 bp, Artificial Sequence
  Tobacco control Real-Time primer-R
tcaccaccagccttctggtaaaca

SEQ ID NO: 11 - DNA, 24 bp, Artificial Sequence
  Arabidopsis control Real-Time primer-F
tttgaccggaaagaccatcaccct

SEQ ID NO: 12 - DNA, 24 bp, Artificial Sequence
  Arabidopsis control Real-Time primer-R
aagacgcaggaccaagtgaagagt

SEQ ID NO: 13 - DNA, 24 bp, Artificial Sequence
  Poplar control Real-Time primer-F
tgcaggcatccacgaaaccacata

SEQ ID NO: 14 - DNA, 24 bp, Artificial Sequence
  Poplar control Real-Time primer-R
ggctagtgctgagatttccttgct